(19) United States
(12) Patent Application Publication
(10) Pub. No.: US 2024/0374170 A1
(43) Pub. Date: Nov. 14, 2024

(54) SYSTEM AND METHOD FOR RF ANALYTE MEASUREMENT GUIDED INSULIN ADMINISTRATION

(71) Applicant: Know Labs, Inc., Seattle, WA (US)

(72) Inventor: John CRONIN, Williston, VT (US)

(21) Appl. No.: 18/654,825

(22) Filed: May 3, 2024

Related U.S. Application Data
(60) Provisional application No. 63/501,523, filed on May 11, 2023.

(51) Int. Cl. A61B 5/145; A61B 5/00; A61B 5/05

(52) U.S. Cl. CPC A61B 5/14532; A61B 5/0022; A61B 5/05; A61B 5/7225; A61B 2560/0219

(57) ABSTRACT

A system for measuring one or more analytes using a real-time, non-invasive radio frequency analyte detection device. The system includes a wearable device attached to a body part, using transmit and receive antennas to send and receive RF signals. The system converts received signals into a digital processor-readable format, compares them with standard waveforms, and utilizes machine learning to identify health parameters. The device or devices measure insulin and glucose levels to determine the effect of insulin administration and predict when insulin will be needed. This data is sent to an admin network which also collects data from a patient or healthcare professional on when insulin is administered and in what dosage. This data is analyzed to recommend when insulin should be administered again and at what dosage.

### FIELD

The present disclosure is generally related to systems and methods of monitoring health parameters and, more particularly, relates to a system and a method of monitoring in real-time at least one analyte level, such as glucose levels, using radio frequency signals.

### BACKGROUND

Individuals with diabetes often struggle to maintain optimal blood glucose levels, as current methods for tracking insulin and glucose levels can be invasive and inconvenient, leading to the need for a more user-friendly and non-invasive approach to monitoring these parameters.

Existing insulin administration and dosage adjustment methods rely on intermittent measurements and may not provide real-time feedback, potentially resulting in suboptimal glycemic control and increased risk of diabetes-related complications.

Healthcare providers and patients require a comprehensive, data-driven solution that can effectively predict insulin needs and personalize dosage recommendations based on individual health parameters to improve overall diabetes management and health outcomes.

Continuous glucose monitors (CGMs) are useful tools for managing diabetes, but they have drawbacks, including cost, sensor accuracy, and sensor lifespan. Users may experience skin irritation or discomfort at the sensor insertion site. CGMs measure glucose in interstitial fluid, which can result in delayed readings compared to actual blood glucose levels.

### DESCRIPTION OF THE DRAWINGS

FIG. 1: Illustrates an example of a radio frequency health monitoring system, according to an embodiment.

FIG. 2: Illustrates an example operation of a Device Base Module, according to an embodiment.

FIG. 3: Illustrates an example operation of an Input Waveform Module, according to an embodiment.

FIG. 4: Illustrates an example operation of a Matching Module, according to an embodiment.

FIG. 5: Illustrates an example operation of a Machine Learning Module, according to an embodiment.

FIG. 6: Illustrates an example operation of a Connection Module, according to an embodiment.

FIG. 7: Illustrates an example operation of an Insulin Log Module, according to an embodiment.

FIG. 8: Illustrates an example operation of a Base Module, according to an embodiment.

FIG. 9: Illustrates an example operation of an Analysis Module, according to an embodiment.

FIG. 10: Illustrates an example operation of a Dosage Module, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

U.S. Pat. Nos. 10,548,503, 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870, U.S. 2022/0151553, are each individually incorporated herein by reference in its entirety.

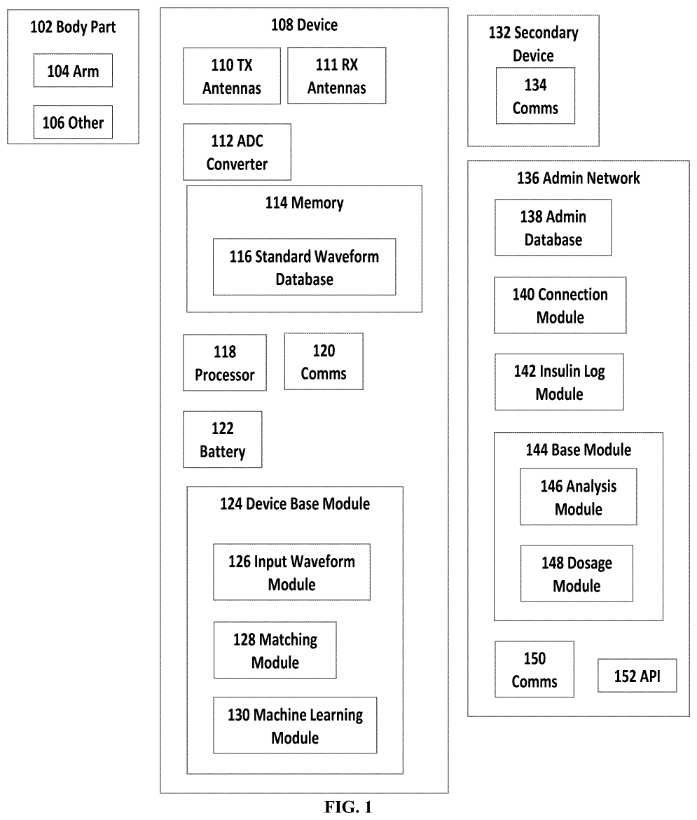

FIG. 1 is a schematic illustration of a system for radio frequency health monitoring. This system is configured to be attached to or in proximity to a body part 102. The body part 102 may be an arm 104. The body part 102 may be another body part 106 besides an arm, such as a leg, finger, chest, head, or any other body part from which useful medical parameters can be taken.

The system may comprise a device 108, which may be a wearable and portable device such as, but not limited to, a cell phone, a smartwatch, a tracker, a wearable monitor, a wristband, and a personal blood monitoring device.

The system may further comprise one or more transmit (“TX”) antennas 110. The one or more TX antennas 110 may be configured to transmit RF in the RF Activated Range from 500 MHZ to 300 GHZ. A pre-defined frequency may correspond to a range suitable for the human body in one embodiment. For example, the one or more TX antennas 110 would transmit radio frequency signals at a range of 120-126 GHz.

The system may further comprise one or more receive (“RX”) antennas 111. The one or more RX antennas 111 may be configured to receive the RF signals in response to the RF signal transmitted from the one or more TX antennas 110.

The system may further comprise an analog-to-digital (AD) converter 112, which may be configured to convert the received RF signals from an analog signal into a digital processor readable format.

The system may further comprise memory 114, which may be configured to store the transmitted RF signals by the one or more TX antennas 110 and store the received portion of the response or responded RF signals from the one or more RX antennas 111. Further, the memory 114 may also store the converted digital processor readable format by the AD converter 112. The memory 114 may include suitable logic, circuitry, and/or interfaces that may be configured to store a machine code and/or a computer program with at least one code section executable by the processor 118. Examples of implementation of the memory 114 may include, but are not limited to, Random Access Memory (RAM), Read Only Memory (ROM), Hard Disk Drive (HDD), and/or a Secure Digital (SD) card.

The system may further comprise a standard waveform database 116, which may contain standard waveforms for known patterns. These may be raw or converted device readings from patients or persons with known conditions. For example, the standard waveform database 116 may include raw or converted device readings from a patient known to have diabetes or an average of multiple patients. This data can be compared to readings from a person with an unknown condition in order to determine if the waveforms taken from that person match any of the known standard waveforms.

The system may further comprise a processor 118, which may facilitate the operation of the device 108 according to the instructions stored in the memory 114. The processor 118 may include suitable logic, circuitry, interfaces, and/or code that may be configured to execute a set of instructions stored in the memory 114.

The system may further comprise a comms 120, which may be a communication element such as a Wi-Fi or RFC transmitter and receiver or physical connection that can send and receive data to other devices of the system and devices external to the system. The comms 120 may be configured to comply with regulatory acts such as HIPPA. For example, the comms 120 may include features such as encryption, access control, audit trails, secure transmission, credential verification, or other features which may protect the integrity and privacy of transmitted or received data.

The system may further comprise a battery 122, which may power hardware modules of the device 108. The device 108 may be configured with a charging port to recharge the battery 122. Charging of the battery 122 may be wired or wireless.

The system may further comprise a device base module 124, which may be configured to store instructions for executing the computer program from the converted digital processor readable format of the AD converter 112. The device base module 124 may be configured to facilitate the operation of the processor 118, the memory 114, the one or more TX antennas 110, the one or more RX antennas 111, and the comms 120. Further, the device base module 124 may be configured to create polling of the RF Activated Range from 500 MHZ to 300 GHZ. It can be noted that the device base module 124 may be configured to filter the RF Activated Range from 500 MHZ to 300 GHZ received from one or more RX antennas 111.

The system may further comprise an input waveform module 126, which may extract a radio frequency waveform from memory. This may be the raw or converted recording of data from the one or more RX antennas 111 from a patient wearing the device. If the entire radio frequency is too long for effective matching, the input waveform module 126 may select a time interval within the data set. This input waveform may then be sent to the matching module 128.

The system may further comprise a matching module 128, which may match the input waveform and each the standard waveforms in the standard waveform database 116 by performing a convolution and/or cross-correlation of the input waveform and the standard waveform. These convolutions and/or cross-correlations are then sent to the machine learning module.

The system may further comprise a machine learning module 130 which has been trained to identify health parameters based on the convolution and/or cross-correlations of the input and standard waveforms. The machine learning module 130 receives the convolutions and cross-correlations from the matching module 128 and outputs any health parameters identified.

The system may further comprise a secondary device 132, which may be another device 108. Each device may measure a separate analyte or the device 108 may measure both analytes.

The system may further comprise a secondary device comms 134, which may be a communication element such as a Wi-Fi or RFC transmitter and receiver or physical connection that can send and receive data to other devices of the system and devices external to the system. The secondary device comms 134 may be configured to comply with regulatory acts such as HIPPA. For example, the secondary device comms 134 may include features such as encryption, access control, audit trails, secure transmission, credential verification, or other features which may protect the integrity and privacy of transmitted or received data.

The system may further comprise an admin network 136, which may be a computer or network of computers which may send and receive data. The admin network 136 may be accessed via an application on a user device such as a PC, smartphone, smartwatch, iPhone, etc. The admin network 136 may be an application center or store which allows secondary devices 132 to be registered to communicate with the network. The admin network 136 may communicate with other networks such as third party networks or other iterations of the admin network 136.

The system may further comprise an admin database 138, which may store the health parameters output by the machine learning module 130 as well as insulin dosage and time data from the insulin log module 142.

The system may further comprise a connection module 140, which may connect to the device 108 and secondary device 132 and collect data which then may be stored in the admin database 138.

The system may further comprise an insulin log module 142, which may allow a patient or healthcare professional to log when insulin was given or taken and in what dosage. This data is then stored in the admin database 138.

The system may further comprise a base module 144, which may initiate the analysis module 146 and dosage module 148. The base module 144 may initiate these modules as soon as there is new insulin log information, when glucose drops below or above a threshold, periodically every hour, when a user of the system requests it, etc.

The system may further comprise an analysis module 146 which runs analytic models such as artificial intelligence (AI) and machine learning (ML) algorithms on the data in the admin database 138. AI and/or ML algorithms, contextual analysis, and predictive analytics are employed to identify patterns, provide personalized insights, and forecast future insulin dosages. This data is then sent to the dosage module 148.

The system may further comprise a dosage module 148, which may recommend to the patient, or their healthcare provider, a dosage of insulin and a time to take or give that dosage. The dosage module 148 may offer multiple recommendations with similar efficacy. For example, taking a 3 unit dosage of insulin immediately may be similarly effective as taking a 5 unit dosage in 1 hour and both these recommendations may be made by the dosage module 148.

The system may further comprise an admin network comms 150, which may be a communication element such as a Wi-Fi or RFC transmitter and receiver or physical connection that can send and receive data to other devices of the system and devices external to the system. The admin network comms 150 may be configured to comply with regulatory acts such as HIPPA. For example, the admin network comms 150 may include features such as encryption, access control, audit trails, secure transmission, credential verification, or other features which may protect the integrity and privacy of transmitted or received data.

The system may further comprise an application programming interface or API 152, which is a set of definitions and protocols for building and integrating application software. The API 152 may be used by programs that want to communicate with the admin network 136. The API 152 may be used to prompt a third party application. The third party application may be a large AI or ML system for data analysis, for example a natural language model such as GPT-4.

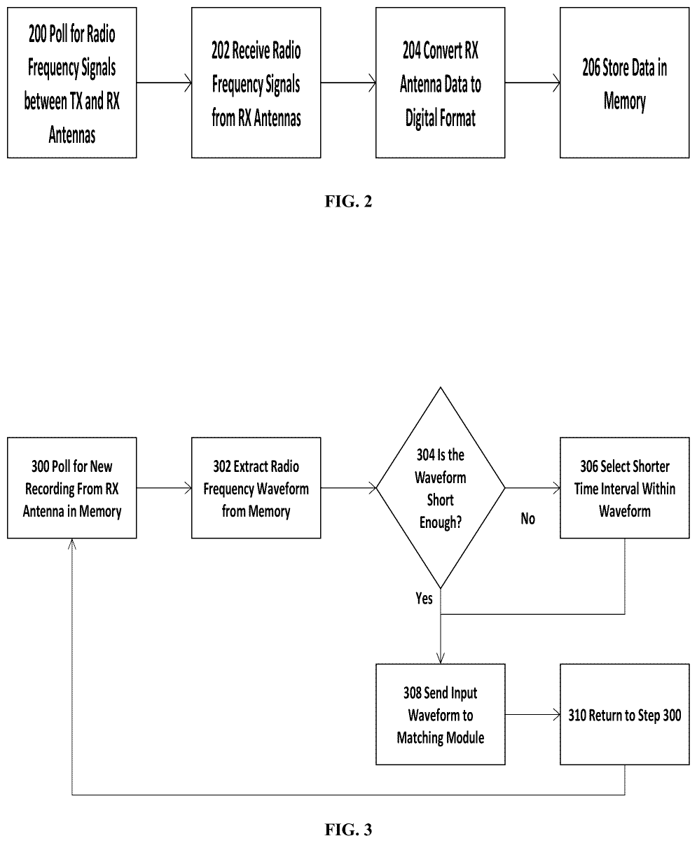

FIG. 2 illustrates an example operation of the device base module 124. The process may begin with the device base module 124 polling the Active Range RF signals between the one or more TX antennas 110 and the one or more RX antennas 111 at step 200. The device base module 124 may be configured to read and process instructions stored in the memory 114 using the processor 118. The one or more TX antennas 110 may be configured to transmit RF in the RF Activated Range from 500 MHZ to 300 GHZ. For example, the one or more TX antennas 110 may use RF signals at a range of 500 MHZ to 300 GHZ. The device base module 124 may receive the RF frequency signals from the one or more RX antennas 111 at step 202. For example, one or more RX antennas 111 receives a response or responded RF of frequency range 300-330 GHz from the patient's blood. The device base module 124 may be configured to convert the received RF signals into a digital format using the AD 112 at step 204. For example, the received RF of 300-330 GHZ frequency range is converted into a 10-bit data signal. The device base module 124 may be configured to store converted digital format into the memory 114 at step 206.

FIG. 3 illustrates an example operation of the input waveform module 126. The process may begin with the input waveform module 126 polling, at step 300, for new recorded data from the one or more RX antennas 111 stored in memory 114. The input waveform module 126 may extract, at step 302, the recorded radio frequency waveform from memory. If there is more than one waveform recorded, the input waveform module 126 may select each waveform separately and loop through the following steps. The input waveform module 126 may determine, at step 304, if the waveform is small enough to be an input waveform for the matching module 128. This will depend on the computational requirements and/or restrictions of the matching module 128. If the waveform is short enough, the input waveform module 126 may skip to step 308. If the waveform is too long, the input waveform module 126 may select, at step 306, a shorter time interval within the entire recorded waveform. For example, if the waveform is 5 minutes long, then only a 30 second interval may be selected. The interval may be selected at random or by a selection process. The input waveform module 126 may send, at step 308, the input waveform to the matching module 128. The input waveform module 126 may return, at step 310, to step 300.

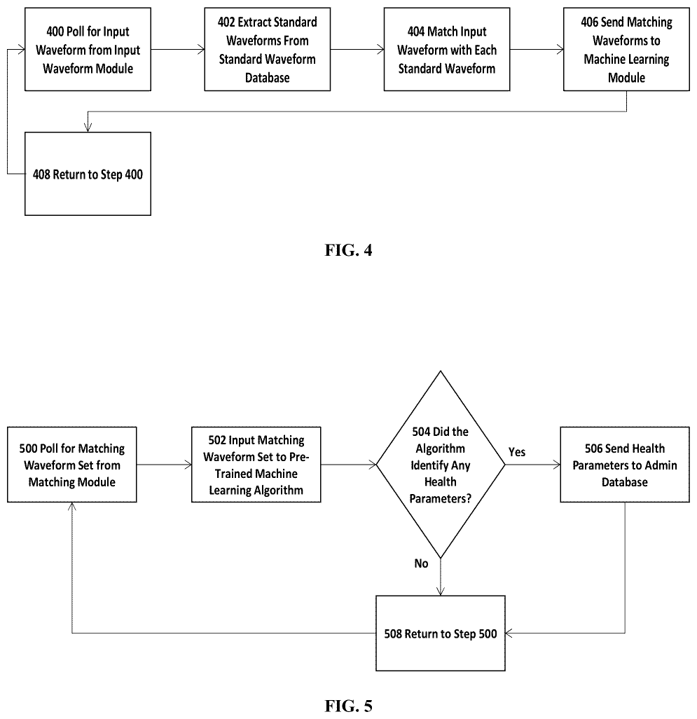

FIG. 4 illustrates an example operation of the matching module 128. The process may begin with the matching module 128 polling, at step 400, for an input waveform from the input waveform module 126. The matching module 128 may extract, at step 402, each standard waveform from the standard waveform database 116. The matching module 128 may match, at step 404, the input waveform with each standard waveform. Matching may be determining which standard waveforms the input waveform is similar to. Matching may involve convolution and/or cross-correlation of the waveforms or any other matching technique. Cross-correlation and convolution are mathematical operations that can be used to determine the similarity between two wave functions. They are often used in signal processing and image recognition applications to find patterns or features in data. Cross-correlation is a measure of the similarity between two signals as a function of the time-lag applied to one of them. It is defined as the integral of the product of two signals after one is flipped and delayed by some amount. By running the cross-correlation function on two wave functions, the output will give a value of similarity between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function which values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 at with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130. The matching module 128 may send, at step 406, the matching waveforms to the machine learning module 130. Matching waveforms may refer to the standard waveforms that were similar to the input waveform, the waveforms that were generated via convolution and/or cross-correlation, or both. The matching module 128 may return, at step 408, to step 400.

FIG. 5 illustrates an example operation of the machine learning module 130. The process may begin with the machine learning module 130 polling, at step 500, for a set of matching waveforms from the matching module 128. Matching waveforms may be a set of standard waveforms that are similar to the input waveform or statistical combinations of the input waveform and standard waveforms, such as convolutions or cross-correlations. The machine learning module 130 may input, at step 502, the set of received waveforms into a pre-trained machine learning algorithm. The machine learning algorithm may be trained on similar sets of matched waveforms where the input waveform is from a patient whose health parameters are known. The waveforms, such as a set of X and Y values, may be input directly into the algorithm. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During the training process, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130 may determine, at step 504, if the algorithm identified any health parameters. Identification may require a certain interval of confidence. For example, if the machine learning algorithm determines that it is more than 70% likely that a health parameter is correct, then that parameter may be considered identified. If multiple conflicting parameters exist, then the most confident may be used. For example, if the algorithm determines that it is 75% likely that the patient's glucose level is between 110-115 mg/dL and 90% likely that the patient's glucose is between 105-110 mg/dL, then the more confident value of 105-110 mg/dL may be identified. If none of the results from the machine learning algorithm are above the confidence threshold, or the results are otherwise inconclusive, the machine learning module 130 may skip to step 508. If any health parameters were identified, the machine learning module 130 may send, at step 506, the health parameters to the admin database 138. The machine learning module 130 may return, at step 508, to step 500.

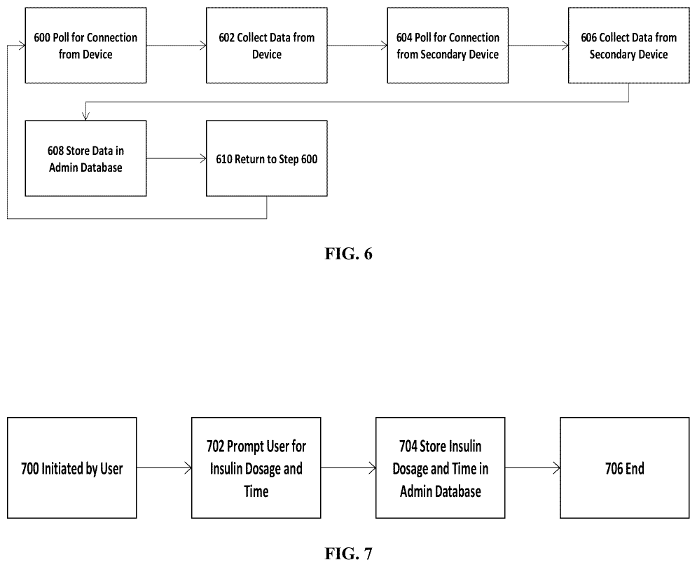

FIG. 6 illustrates an example operation of the connection module 140. The connection module 140 may poll, at step 600, for a connection from the device 108. If no connection is detected, the connection module 140 may search for a device 108 that is attempting to connect or may skip to step 604. The connection module 140 may collect, at step 602, data from the device 108. This may be real-time data or periodically updated data. This data may come directly from the machine learning module 130. The connection module 140 may poll, at step 604, for a connection from the secondary device 132. If no connection is detected, the connection module 140 may search for a secondary device 132 that is attempting to connect or may skip to step 608. The connection module 140 may begin at this step if a secondary device 132 is unavailable or unnecessary. The connection module 140 may collect, at step 606, data from the secondary device 132. This may be real-time data or periodically updated data. The connection module 140 may store, at step 608, the data from the device 108 and/or secondary device 132 in the admin database 138. The connection module 140 may add contextual data such as the ID of the secondary device 132, the ID of the device 108, the analyte measured by the device 108, the time data was retrieved, etc., to the received data before storage in the admin database 138. The connection module 140 may return, at step 610, to step 600.

FIG. 7 illustrates an example operation of the insulin log module 142. The insulin log module 142 may initiate, at step 700, the process upon receiving input from a user. For example, the user may select “log insulin dosage” from a menu on a webpage or within an application. Users may refer to a patient and/or their healthcare professional. The insulin log module 142 may prompt, at step 702, the user for insulin dosage and time. The insulin log module 142 may store, at step 704, the insulin dosage and time in the admin database 138. The insulin log module 142 may end at step 706.

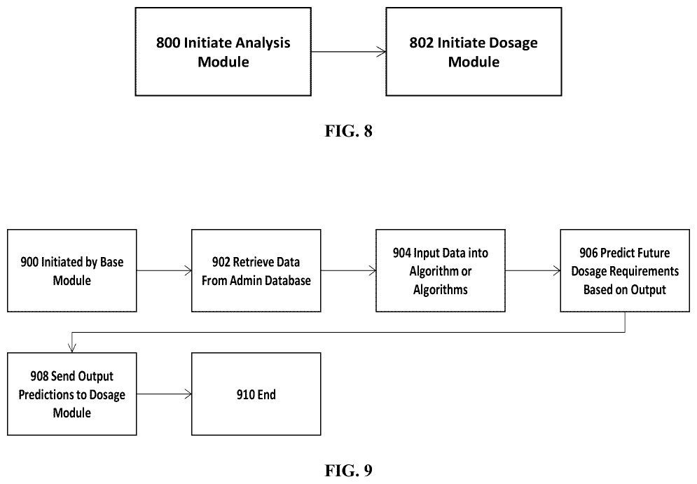

FIG. 8 illustrates an example operation of the base module 144. The base module 144 may initiate, at step 800, the analysis module 146, which may analyze the patient's insulin and glucose levels, dosage of insulin taken, and time of insulin taken. The analysis module may determine the efficacy of the insulin taken and determine when insulin should be taken again and at what dosage. The base module 144 may initiate, at step 802, the dosage module 148, which may recommend insulin dosages and intake times to the patient and/or their healthcare provider based on the analysis from the analysis module 146.

FIG. 9 illustrates an example operation of the analysis module 146. The analysis module 146 may be initiated, at step 900, by the base module 144. The analysis module 146 may access, at step 902, the admin database 138 to retrieve relevant data for analysis. The data retrieved may include but is not limited to, insulin levels, glucose levels, insulin dosage amounts, and the corresponding timestamps of insulin administration. The analysis module 146 may input, at step 904, the retrieved data, along with any relevant contextual information, into one or more specialized AI and/or ML algorithms designed to estimate optimal insulin administration times and dosages for the patient. These algorithms may consider factors such as insulin levels, glucose levels, insulin dosage history, and patient-specific information such as demographics, medical history, lifestyle habits, and genetic predispositions if available. The algorithm or algorithms may be a simple mathematical formula that compares insulin dosage to insulin and glucose level. For example, a 3-unit insulin injection converts almost entirely to insulin, which immediately reduces glucose levels by 50 mg/dL, but glucose climbs at a rate of 10 mg/dL per hour. Then the analysis module 146 may output that a 3-unit insulin injection will be needed again in 5 hours, or that a 2-unit insulin injection may be needed in roughly 3.5 hours to keep glucose from climbing too high. The algorithm or algorithms may involve more advanced analysis such as linear regression, multivariable differentials, cross-correlation, etc., and may need to update the predictions periodically. The algorithm or algorithms may be machine learning algorithms or neural networks which have been trained on a corpus of inputs and outputs. The analysis module 146 may predict, at step 906, future dosage requirements based on the output from the algorithm or algorithms, taking into account the patient's unique circumstances and glycemic control requirements. These algorithms may generate multiple insulin administration options, each consisting of a recommended dosage and corresponding time for administration, allowing healthcare providers and patients to make informed decisions regarding their insulin management. For example, when a patient's blood sugar is high, the algorithm or algorithms may provide options such as administering 3 units of insulin immediately, followed by another 3 units after 2 hours, or administering 5 units of insulin in 1 hour. In another example, a patient who is planning to engage in physical activity may receive recommendations for adjusting insulin administration, such as reducing the dosage by 1 or 2 units before exercise to prevent hypoglycemia. Dosage and administration time options may be limited. For example, the algorithm may not output dosages below 1 unit or above 10 units and may round to the nearest unit. The analysis module 146 may leverage the capabilities of external AI algorithms, such as GPT-4, via the API 152 to further enhance the analysis and provide more refined and accurate insulin administration estimations. For example, by incorporating GPT-4's natural language processing capabilities, the system can analyze and interpret complex medical literature, enabling the AI algorithms to consider the latest diabetes management research and guidelines when generating insulin administration recommendations. The analysis module 146 may send, at step 908, the output data from the algorithm or algorithms in step 906 to the dosage module 148. The analysis module 146 may end at step 910.

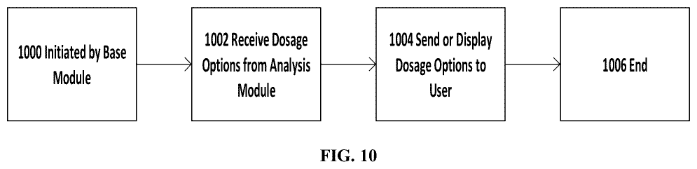

FIG. 10 illustrates an example operation of the dosage module 148. The dosage module 148 may be initiated, at step 1000, by the base module 144. The dosage module 148 may receive, at step 1002, dosage options from the analysis module 146, which include recommended insulin dosages and corresponding times for administration. The dosage module 148 may send or display, at step 1004, the dosage options to the user through various means, facilitating informed decision-making by the patient or their healthcare provider concerning insulin administration. The dosage options may be presented on a user interface, such as a graphical display or touchscreen, incorporated within the device 108, or a separate, connected device like a smartphone, tablet, or computer. The interface may utilize visual elements like charts, graphs, or text to clearly convey the recommended insulin dosages and corresponding administration times. The dosage module 148 may send the dosage options to the user via electronic communication methods, such as email, SMS, or instant messaging applications. The electronic communication may contain the recommended insulin dosages and corresponding administration times, along with any supplementary information or contextual data that may assist the patient or healthcare provider in making an informed decision. The user may be able to indicate what time they intend to take the next dosage or what dosages are available, in which case the options may be filtered to meet those criteria, or the analysis module 146 may be rerun with those output constraints. The dosage module 148 may end at step 1006.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A real-time health monitoring system, comprising:
a real-time, non-invasive radio frequency (RF) analyte detection device having at least one transmit antenna and at least one receive antenna; the at least one transmit antenna is positioned and arranged to transmit an RF signal into a user, and the at least one receive antenna is positioned and arranged to detect an RF response signal resulting from transmission of the RF signal by the at least one transmit antenna into the user;
an admin network that is in communication with the real-time, non-invasive RF analyte detection device; and
wherein the system is configured to:
control the real-time, non-invasive RF analyte detection device to collect real-time insulin data of the user using the at least one transmit antenna and the at least one receive antenna;
control the real-time, non-invasive RF analyte detection device to collect real-time glucose data of the user using the at least one transmit antenna and the at least one receive antenna;
prompt, using the admin network, entry of a user actual insulin dosage and insulin administration time combination;
determine, using the admin network, a user future insulin dosage and insulin administration time combination based on the collected real-time insulin data, the collected real-time glucose data, and the user actual insulin dosage and insulin administration time combination; and
present the user future insulin dosage and insulin administration time combination.

2. The real-time health monitoring system of claim 1, wherein the real-time, non-invasive RF analyte detection device further includes an analog-to-digital converter connected to the at least one receive antenna; a memory connected to the analog-to-digital converter that stores data therefrom; a rechargable battery; and a communication element that allows the real-time, non-invasive RF analyte detection device to communicate with the admin network.

3. The real-time health monitoring system of claim 1, further comprising a secondary device with a communication element that allows the secondary device to communicate with the admin network.

4. The real-time health monitoring system of claim 1, wherein the admin network includes an insulin log module that is configured to prompt for the user actual insulin dosage and insulin administration time combination.

5. A real-time health monitoring method, comprising:
collecting real-time insulin data of a user using at least one transmit antenna and at least one receive antenna of a real-time, non-invasive radio frequency (RF) analyte detection device by using the at least one transmit antenna to transmit an RF signal into the user, and using the at least one receive antenna to detect an RF response signal resulting from transmission of the RF signal by the at least one transmit antenna into the user;
collecting real-time glucose data of the user using the at least one transmit antenna and the at least one receive antenna of the real-time, non-invasive RF analyte detection device by using the at least one transmit antenna to transmit another RF signal into the user, and using the at least one receive antenna to detect another RF response signal resulting from transmission of the another RF signal by the at least one transmit antenna into the user;
collecting an actual insulin dosage and insulin administration time combination of the user;
determining a user future insulin dosage and insulin administration time combination based on the collected real-time insulin data, the collected real-time glucose data, and the actual insulin dosage and insulin administration time combination; and
presenting the user future insulin dosage and insulin administration time combination.

6. The real-time health monitoring method of claim 5, comprising converting the RF respone signal and the another RF response signal from analog to digital, and storing the digital signals in a memory.

7. The real-time health monitoring method of claim 5, comprising prompting for entry of the actual insulin dosage and insulin administration time combination.

8. The real-time health monitoring method of claim 5, comprising collecting data from a secondary device that is separate from the real-time, non-invasive RF analyte detection device.
